(19) United States
(12) Patent Application Publication
(10) Pub. No.: US 2005/0241562 A1
(43) Pub. Date: Nov. 3, 2005

(54) Foot brace

(76) Inventors: Mark Nysether, Arlington, WA (US); Matthias Poischbeg, Everett, WA (US); R. Lee Rawls, Woodinville, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/079,915

(22) Filed: Mar. 8, 2005

Related U.S. Application Data
(60) Provisional application No. 60/551,114, filed on Mar. 8, 2004.

(51) Int. Cl. A61F 5/00; B63B 35/00; B63B 35/71; B63B 17/00

(52) U.S. Cl. 114/347; 114/363

(57) ABSTRACT

A kayak user braces his or her feet against footrests that project inward from the sides of the kayak. The positions of the footrests can be adjusted to accommodate users of different heights, and the footrests can be pivoted to control the position of the rudder of the kayak.

### CROSS-REFERENCE TO RELATED APPLICATION

This application claims the benefit of U.S. Provisional Application No. 60/551,114, filed on Mar. 8, 2004.

### FIELD OF THE INVENTION

The present invention pertains to a foot brace having a footrest adjustable toward and away from a user and conveniently lockable in a selected adjusted position.

### BACKGROUND OF THE INVENTION

In a typical personal watercraft, such as a kayak, a user braces his or her feet against footrests that project inward from the sides of the craft. Often the positions of the footrests can be adjusted to accommodate users of different heights. See, for example, U.S. Pat. No. 4,942,840, which shows an adjustable foot brace. In some applications, the footrests are connected to a rudder assembly to be used in steering the craft.

### SUMMARY OF THE INVENTION

A control rod extends lengthwise of an elongated slide channel mounted in a personal watercraft such as a kayak. A base unit having an inward projecting footrest is slidable along the slide channel. The control rod has longitudinally spaced teeth which will interfit with slots of the base unit to lock the base unit and footrest in a selected longitudinal position. The control rod has an extended handle portion within easy reach of a user for locking and unlocking the base unit and footrest. The footrest can be a pedal that swings to control the position of a rudder.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagrammatic perspective of a foot brace in accordance with the present invention as mounted in a watercraft, namely, a kayak.

FIG. 2 is an interior side, rear perspective of the foot brace removed from the kayak.

FIG. 3 is an interior side, front perspective of the foot brace removed from the kayak.

FIG. 4 is an exterior side, rear perspective of the foot brace removed from the kayak.

FIG. 5 is a bottom plan of the foot brace.

FIG. 6 is a top plan of the foot brace.

FIG. 7 is an interior side elevation of the foot brace.

FIG. 8 is an exterior side elevation of the foot brace.

FIG. 9 is an enlarged, fragmentary perspective of the leading end portion of the foot brace.

FIG. 10 is a diagrammatic perspective of a second embodiment of a foot brace in accordance with the present invention as mounted in a kayak.

FIG. 11 is an interior side, front perspective of the foot brace of FIG. 10 removed from the kayak.

FIG. 12 is a perspective corresponding to FIG. 11. with some parts shown in exploded relationship.

FIG. 13 is a diagrammatic interior side elevation of the second embodiment of foot brace with parts shown in different adjusted positions.

### DETAILED DESCRIPTION OF THE PREFERRED EMBODIMENT

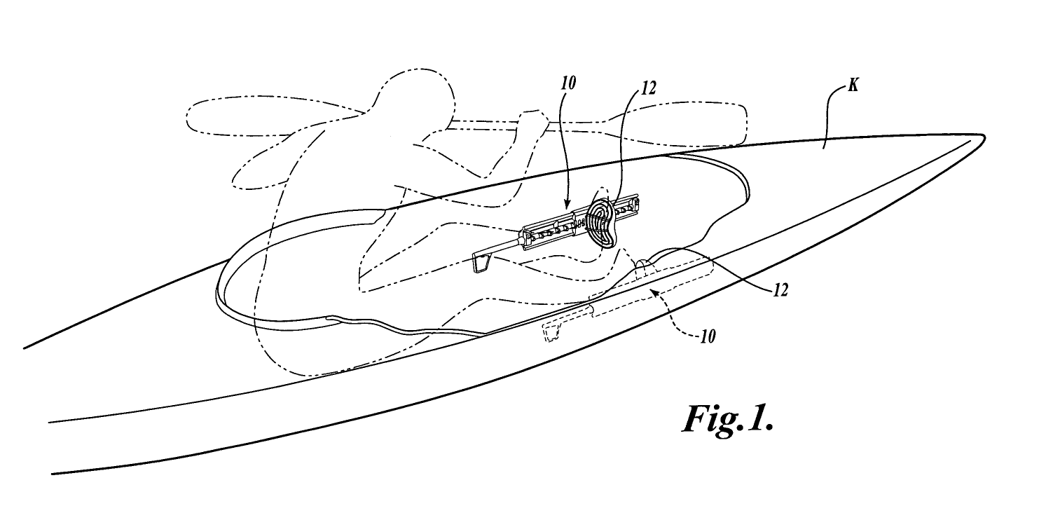

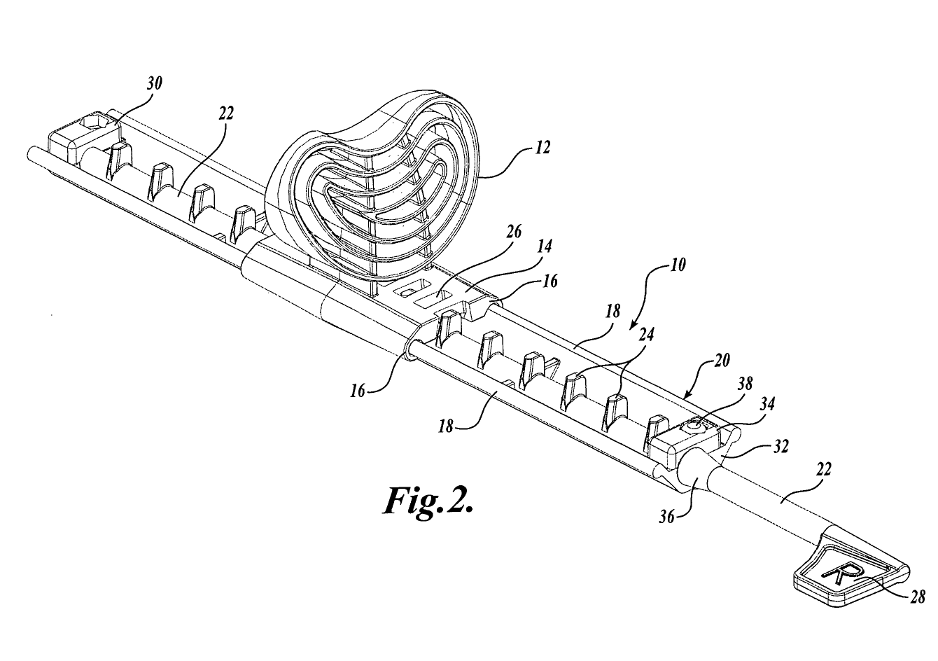

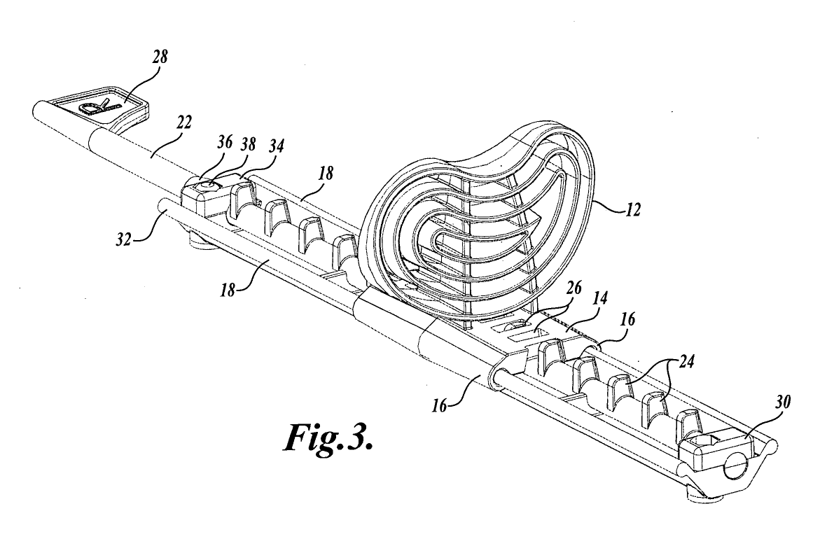

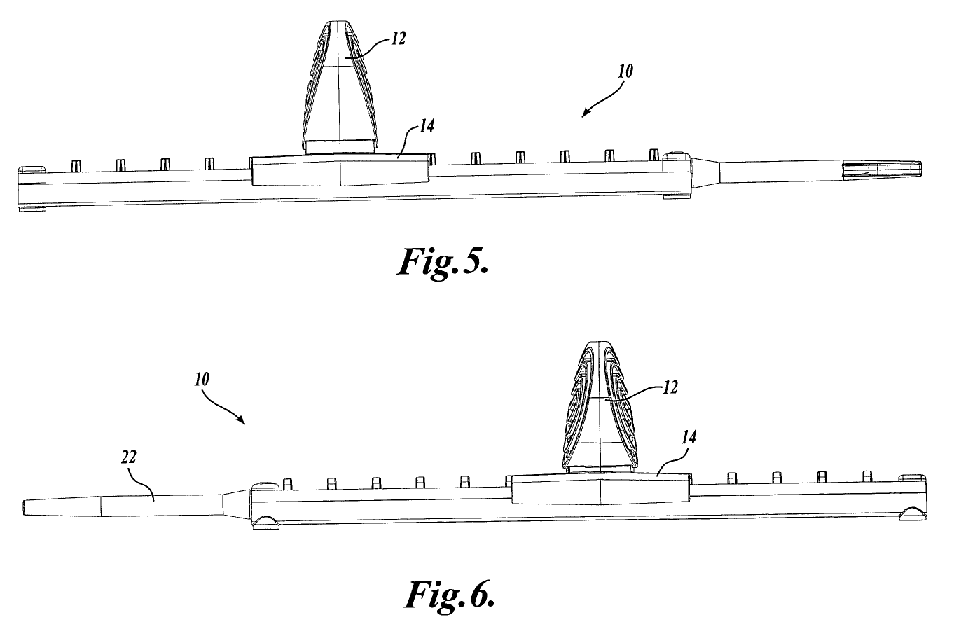

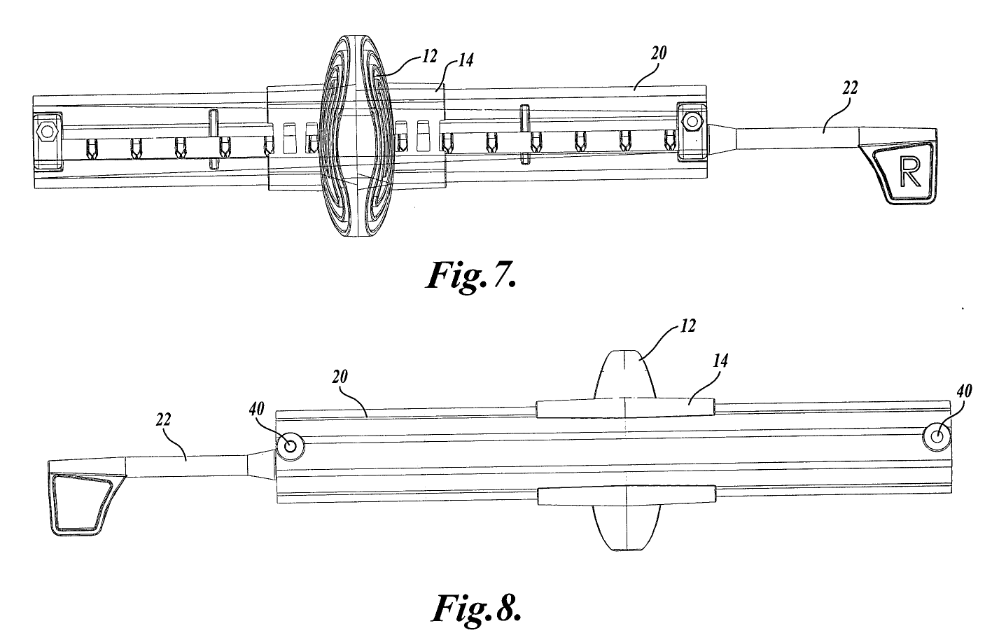

FIG. 1 is a diagrammatic perspective of a foot brace 10 in accordance with the present invention as mounted in a watercraft, namely, a kayak K. Brace 10 has a footrest 12 that is adjustable fore and aft. The footrest can be locked in a desired adjusted position. Two such foot braces are provided, one at the left (port) and one at the right (starboard), the right foot brace being illustrated in broken lines at the bottom of FIG. 1. The right foot brace is shown in detail in FIGS. 2-8. The left foot brace is the mirror image of the right.

Referring to FIGS. 2-8, the footrest 12 projects from an integral base 14 having hooked sides 16 for guiding the base and footrest along top and bottom rails 18 of an elongated slide channel 20. A long control rod 22 extends through the channel 20 and has regularly spaced latch teeth 24. In the locked position illustrated in FIG. 2, the latch teeth interengage with slots 26 in the base 14 of the footrest, to lock the footrest in a selected position.

The control rod 22 is rotatable by manipulation of an external control handle 28 which is oriented so as to be spaced from the slide channel 20 in the direction of the user. Thus, the user can conveniently reach down to manipulate the control handle. The control rod 22 has a forward end journaled between the forward end of the channel 20 and a transverse cap 30. Similarly, the rearward end portion of the control rod 22 is journaled in the rear or aft end 32 of the channel and a cap 34. An enlargement 36 on the control rod prevents longitudinal forward sliding of the rod by engagement against the cap 34 and channel end 32, and the adjacent latch tooth 24 prevents aftward sliding.

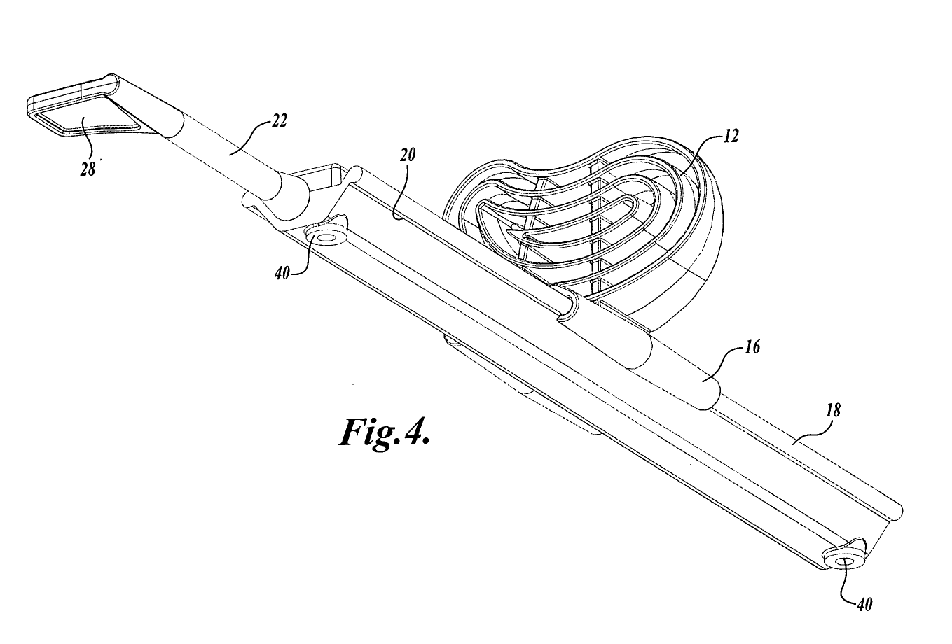

Caps 30 and 34 are clamped to their corresponding channel ends by fasteners 38 which can extend through the channel and into mounting structure of the watercraft. For example, FIG. 4 shows the channel openings 40 which are aligned with the respective openings of the cap pieces 30 and 34 such that fasteners 38 of appropriate size project through the channel and secure the assembly in the watercraft.

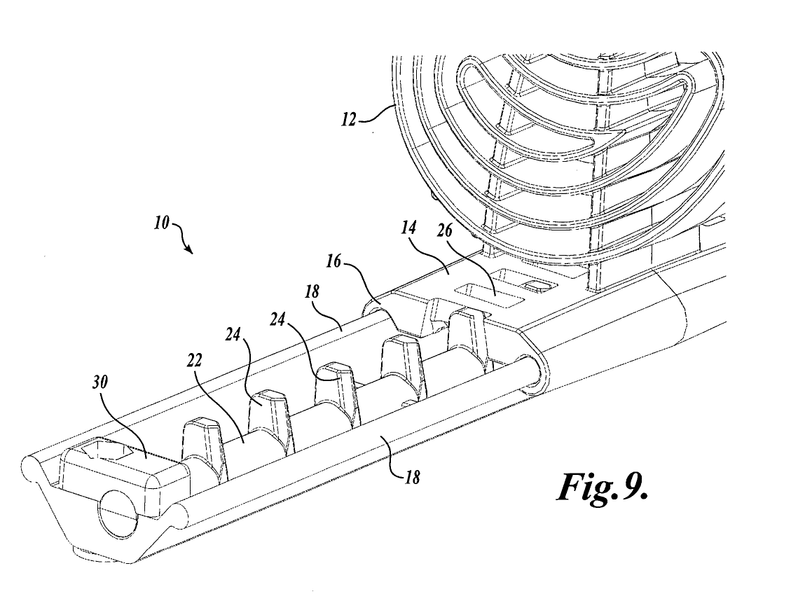

To unlock the footrest 12 and allow longitudinal sliding movement thereof, the user need only rotate the control handle 28 90°, counterclockwise as viewed in FIG. 2, to disengage the latch teeth 24 from the slots 26. The user then can slide the footrest to the desired position and rotate the control handle 90° in the other direction to reengage selected latch teeth with the slots 26, and lock the footrest 12 in the new position. Preferably, the bottom edge portions of the latch piece are beveled, as best seen in FIG. 9, to assist in guiding the latch teeth into the nearest slot. The incremental adjustment permitted by the mechanism is determined by the space between adjacent slots 26. In the illustrated embodiment, the latch teeth are spaced apart a distance exactly twice the distance between adjacent slots, so that a latch tooth engages in every other slot.

All parts can be formed of rigid plastic, except the fasteners which can be screws or bolts.

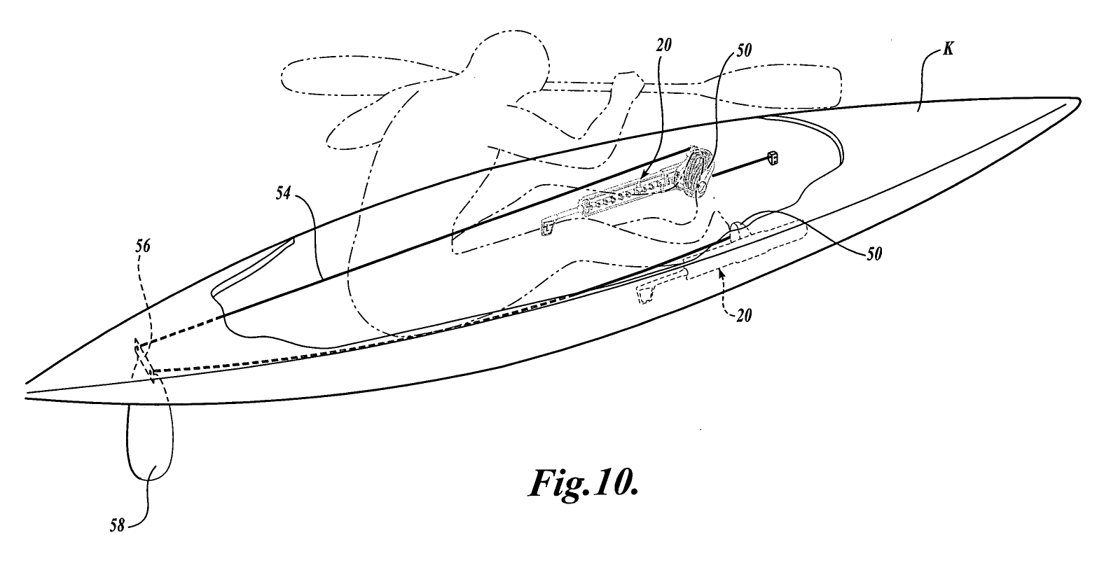

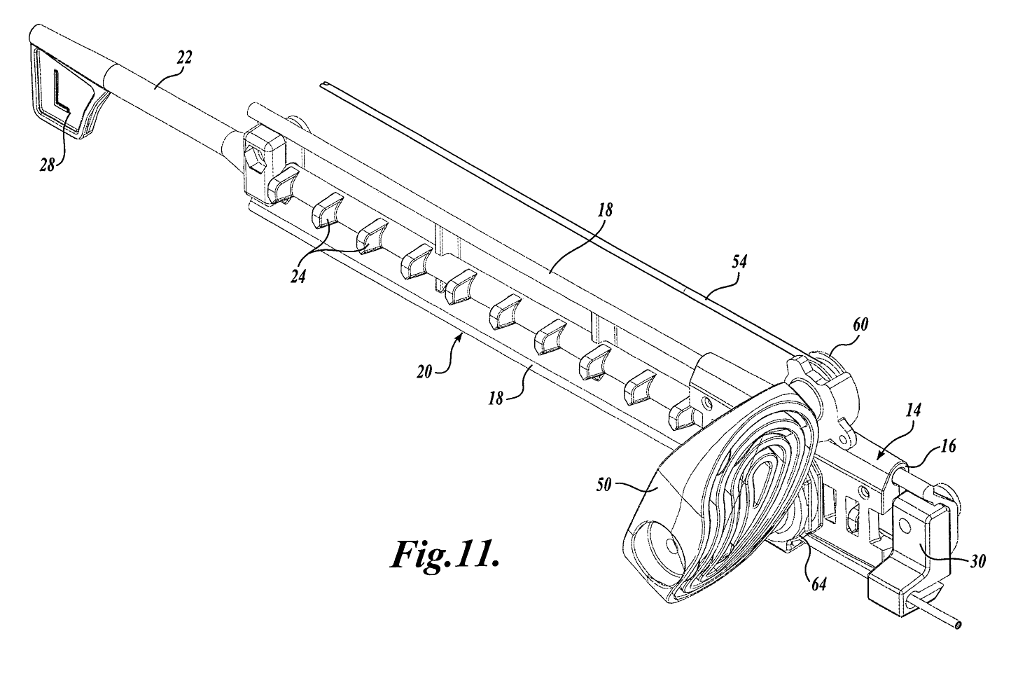

FIGS. 10-13 illustrate a second embodiment of a foot brace in accordance with the present invention, having modifications that permit rudder control. Many of the parts are the same or essentially the same as the embodiment described above, and are similarly numbered. With reference to FIG. 11, these include the control rod 22 with its control handle 28 and latch teeth 24. The control rod extends through the slide channel 20, and the latch teeth 24 selectively interengage with the base 14 which has hooked sides 16 for guiding the base along top and bottom rails 18 of the slide channel.

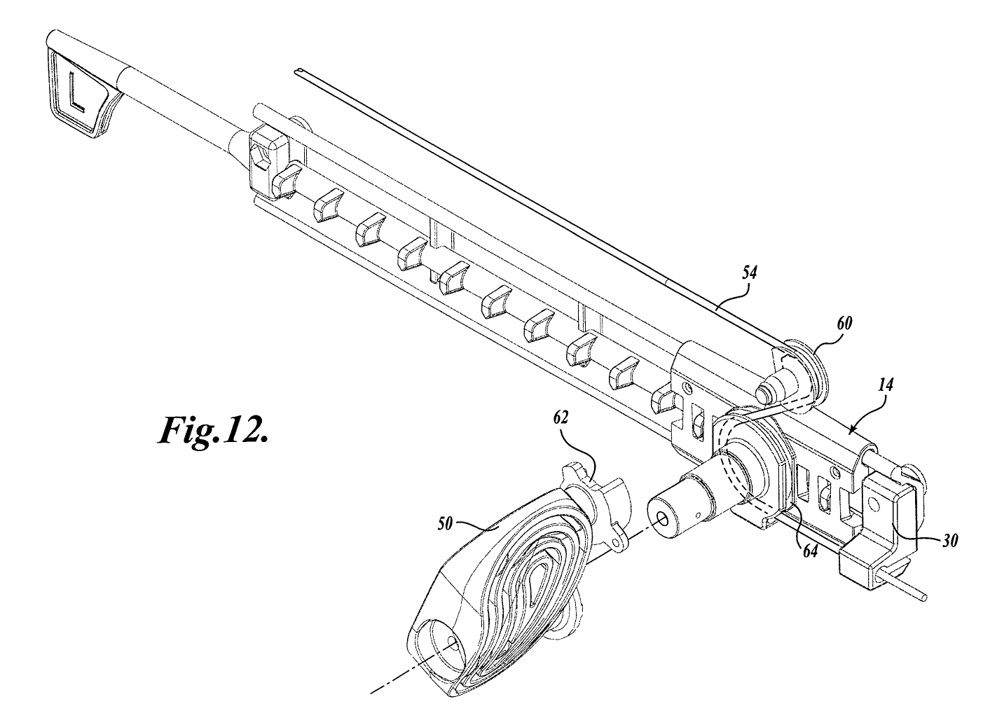

A footrest is in the form of a pedal 50 that swings about a horizontal axis relative to the base 14, rather than being stationarily mounted on the base as in the previously described embodiment. For example, FIG. 12 shows the base 14 and inward projecting mounting stud 52 received in a bore toward the bottom of the pedal 50. Thus, the top portion of the pedal can be swung fore and aft by the user.

FIG. 12 also illustrates the routing of a control cable 54 for a rudder. With reference to FIG. 10, control cable 54 connects at its rear end to a control arm 56 for the rudder 58. A similar control cable connects at the other side. Manipulation of the control cables adjusts the position of the rudder. More specifically, as one cable is shifted forward, the other is shifted rearward, and the rudder will turn.

Returning to FIG. 12, the leading end portion of the control cable 54 extends around a pully 60 rotatably carried at the upper portion 62 of the pedal 50. From pully 60 the control cable 54 extends rearward and around another pully 64 rotatably mounted on the sliding base 14. The axis of pully 64 is the same as the swinging axis for the pedal. From the second pully 64, the control cable extends to and is anchored in the leading end cap 30.

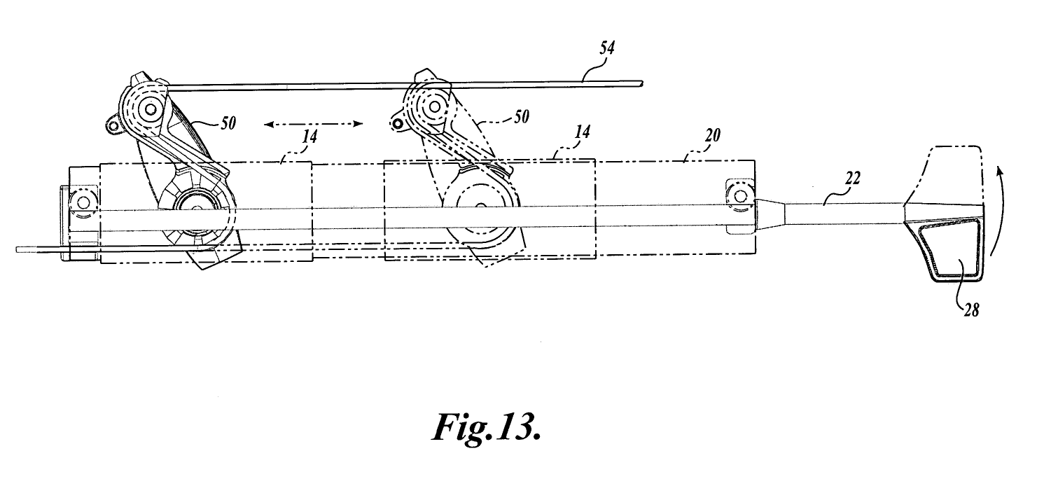

With reference to FIG. 13, the longitudinal position of the base 14 and, consequently, the pedal 50 can be adjusted fore and aft of the channel 20 as for the previously described embodiment, by manipulation of the control handle 28 and control rod 22 and intertitting the slots of the base with selected latch key of the control rod. This aspect is identical to the aspect previously described. Also, the position of the rudder control cable 54 is not affected so long as the pedal is not rotated. Tension of the cable remains the same as the length of the upper stretch increases and the length of the lower stretch decreases. From any position in which the base 14 is locked, the position of the rudder can be controlled by adjusting the angle of the pedals 50. With reference to FIG. 13, if the pedal is tilted to the left, i.e., forward, tension is applied to the control cable which tends to swing the rudder as well as swing the other pedal oppositely. Thus, the embodiment of FIGS. 10-13 allows both longitudinal adjustment of the pedals and control of the rudder position.

While the preferred embodiment of the invention has been illustrated and described, it will be appreciated that various changes can be made therein without departing from the spirit and scope of the invention.

1. A foot brace for a watercraft comprising:
an elongated slide channel mountable in a watercraft;
a base unit slidable along the channel;
a footrest carried by the base unit; and
a control rod extending lengthwise of the channel and rotatably mounted therein, the control rod and channel having cooperating members that interfit and lock the base unit to the slide channel when the control rod is in a first rotated position, the cooperating members being freed from each other to permit relative movement of the bace unit and slide channel when the control rod is rotated from the first rotated position to a second rotated position, the control rod having a handle portion extending beyond the slide channel for manipulation by a user.

2. The foot brace defined in claim 1, in which the footrest is a pedal interconnected with a rudder control cable constructed and arranged relatively such that longitudinal movement of the base unit and pedal do not affect the tension of the control cable but rotary movement of the pedal and base unit does affect the tension and position of the control cable.
